(19) United States
(12) Patent Application Publication
(10) Pub. No.: US 2009/0247941 A1
(43) Pub. Date: Oct. 1, 2009

(54) NASAL IRRIGATOR FOR YOGA WITH A CONNECTION MECHANISM

(76) Inventor: SHIN-CHIANG LU, Taipei (TW)

Correspondence Address: Dr. BANGER SHIA, 102 Lindencrest Ct., Sugar Land, TX, 77479-5201, US

(21) Appl. No.: 12/055,276

(22) Filed: Mar. 25, 2008

(51) Int. Cl. A61M 31/00

(52) U.S. Cl. 604/73; 604/94.01; 604/275

(57) ABSTRACT

A nasal irrigator for yoga includes a connection mechanism, an air vent and a conduit. The connection mechanism is used to connect a vessel. The liquid of the vessel flows to the nasal irrigator and the air vent will balance the pressure between the vessel and outside during the flowing. Hence, the user can rinse the nasal cavity by himself easily. Also, the nasal irrigator is convenient to carry.

### BACKGROUND OF THE INVENTION

1. Field of Invention

The present invention relates to a nasal irrigator, and more particularly to a nasal irrigator for yoga to rinse the nasal cavity by himself easily and is convenient to carry.

2. Related Art

Yoga as we all know it is aimed to unite the mind, the body, and the spirit. Yoga therefore is considered therapeutic. It helps you become more aware of your body's posture, alignment and patterns of movement. It makes the body more flexible and helps you relax even in the midst of a stress stricken environment.

Yoga is a form of postures and exercises practiced to achieve control and balance of the body and mind and spirit along with holistic health and fitness. Yoga is good for what ails you. Specifically, research shows that yoga helps manage or control anxiety, arthritis, asthma, back pain, blood pressure, carpal tunnel syndrome, chronic fatigue, depression, diabetes, epilepsy, headaches, heart disease, multiple sclerosis, stress and other conditions and diseases.

Substantial research has been conducted to look at the Health Benefits of Yoga—from the Yoga Postures (Asanas), Yoga Breathing (Pranayama), and Meditation. During Meditation or Pranayama, the yoga player usually uses some auxiliaries, such as neti pot or nasal rinser, to rinse of the nose alleviate and reduce troubles with allergies, since the rinsing flushes away allergens (allergy-forming substance), microbes (virus, bacteria), bi-products of the body (pus, mucus) and dust and soot particles.

Due to the neti pot or nasal rinser has to with the rinsing fluid to clean the nasal cavity, it has a container with a predetermine volume to store the rinsing fluid. Hence, the neti pot or nasal rinser is too big or heavy to carry.

### SUMMARY OF THE INVENTION

To solve the technical problems existing in the prior art, the present invention provides a nasal irrigator for yoga. The nasal irrigator for yoga is easy to use and convenient to carry.

Accordingly, the present invention is directed to a nasal irrigator for yoga that includes a body, a connection mechanism and two tubes. The body has two sides. The connection mechanism extends from one side of the body and selectively connected to a vessel with a rinsing liquid therein. The two tubes are mounted on the other side of the body and inclined to opposite sides to define an air vent and a conduit. The rinsing liquid of the vessel flows to the body and the conduit for cleaning a nasal cavity of a yoga player, and during the flowing the air vent balances a pressure inside the vessel. Hence, the user can rinse the nasal cavity by himself easily. Also, due to the nasal irrigator has the connection mechanism to connect any vessels or bottles, the yoga player need not to take a big or heavy vessel. The nasal irrigator is convenient to carry.

It is to be understood that both the foregoing general description and the following detailed description are exemplary, and are intended to provide further explanation of the invention as claimed.

### BRIEF DESCRIPTION OF THE DRAWINGS

The present invention will become more fully understood from the detailed description given hereinbelow illustration only, and thus are not limitative of the present invention, and wherein:

FIG. 1 is a perspective view of a nasal irrigator for yoga in accordance with first embodiment of the present invention.

FIG. 2 is a perspective view of a nasal irrigator when connecting to a vessel in accordance with second embodiment of the present invention.

FIG. 3 is a perspective view of a nasal irrigator when cleaning the nasal cavity of a yoga player in accordance with third embodiment of the present invention.

FIG. 4 is a perspective view of a nasal irrigator for yoga in accordance with second embodiment of the present invention.

### DETAILED DESCRIPTION OF THE INVENTION

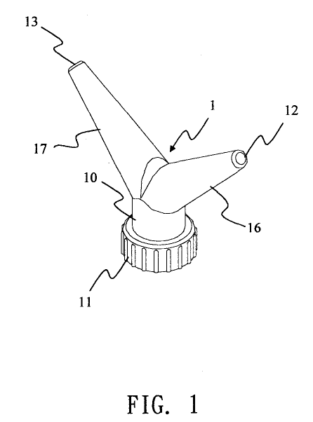

The present invention is related to a nasal irrigator for yoga. Please refer to FIG. 1, a perspective diagram of a nasal irrigator for yoga in accordance with first embodiment of the present invention.

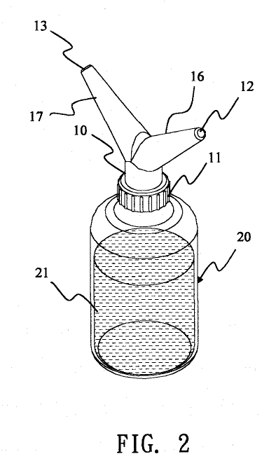

The nasal irrigator 1 for yoga that includes a body 10, a connection mechanism 11 and two tubes, a first tube 16 and a second tube 17. The body 10 has two sides. The connection mechanism 11 extends from one side of the body 10 and selectively connected to a vessel 20 with a rinsing liquid 21 therein, shown in FIG. 2. The first tube 16 and the second tube 17 are mounted on the other side of the body 10 and inclined to opposite sides to define an air vent 12 and a conduit 13. The rinsing liquid 21 of the vessel 20 flows to the body 10, and during the flowing the air vent 12 balances a pressure between inside the vessel 20 and outside. Hence, the rinsing liquid 21 can flow smoothly.

Please refer to FIG. 2, a perspective view of a nasal irrigator when connecting to a vessel in accordance with second embodiment of the present invention.

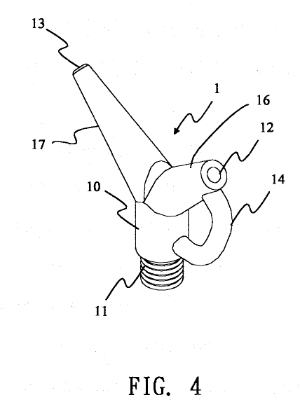

The connection mechanism 11 is used to connect the vessel 20, which is available around the user, such as PET bottle with potable water, mineral water or distilled water, or saline solution. Due to these vessels 20 are easy to obtain, the yoga player just need take the nasal irrigator 1. For connecting to the vessel 20 easily, the connection mechanism 11 is designed to have the same size or dimension. And the connection mechanism 11 and connects to the vessel 20 by screw-fastening, see FIG. 1, mortising or the connection mechanism 11 is a bung, see FIG. 4, and any other connecting way. After connecting, the yoga player can clean nasal cavity by the rinsing fluid 21 inside the vessel 20, such as potable water, mineral water, distilled water or saline solution. Due to the rinsing fluid 21 is supplied from outside resource, the body 10 of the nasal irrigator 1 need not to have big or huge volume. Hence, the nasal irrigator 1 is easy to carry for the yoga player.

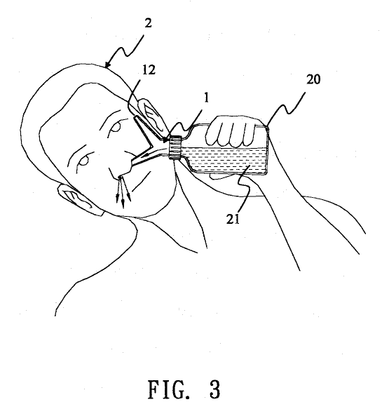

Before using the nasal irrigator 1, the user can mix some sea salt into the water of the vessel 20 or just use the saline solution. It is preferable that the percentage of the salt matches the osmotic pressure of the tissue fluid. And the temperature of the rinsing fluid 21 can not too high or low to prevent to irritate the nasal cavity. Please refer to FIG. 3, the nasal irrigator 1 connects to the vessel 20 by the connection mechanism 11. Put the front end of the second tube 17 into the nostril of the yoga player 2 and the head of the yoga player leans forward in 15 degrees. If cleaning the right nostril first, the head of the yoga player need to turn right with an angle of 45 degree to the left shoulder, and hold the breath. The rinsing fluid 21 will flows from the vessel 20 to body 10, the second tube 17 and outside to the right nostril from the conduit 13. The rinsing fluid 21 would flows from the right nostril through the passage between the two nostrils, and flows out from the left nostril. During the rinsing fluid 21 flows, the air flows into the vessel 20 from the air vent 12 to balance the pressure between inside the vessel 20 and outside. If the user can not hold the breath, he can open mouth to breath and finish the cleaning the nasal cavity. And cleaning the left nostril is the same way.

Please refer to FIG. 4, a perspective view of a nasal irrigator for yoga in accordance with second embodiment of the present invention.

The body 10 further includes a hand grip 14. The hand grip 14 extends from the body 10 to the first tube 16, which is with the air vent 12. The yoga player can grasp the hand grip 14 for cleaning the nasal cavity smoothly.

The invention being thus described, it will be obvious that the same may be varied in many ways. Such variations are not to be regarded as a departure from the spirit and scope of the invention, and all such modifications as would be obvious to one skilled in the art are intended to be included within the scope of the following claims.

What is claimed is:

1. A nasal irrigator for yoga, comprising:
a body, having two sides;
a connection mechanism, extended from one side of the body and selectively connected to a vessel with a rinsing liquid therein; and
two tubes, mounted on the other side of the body and inclined to opposite sides to define an air vent and a conduit;
whereby the rinsing liquid of the vessel flows to the body and the conduit for cleaning a nasal cavity of a yoga player, and during the flowing the air vent balances a pressure inside the vessel.

2. The nasal irrigator of claim 1, wherein the body further comprises a hand grip.

3. The nasal irrigator of claim 2, wherein the hand grip extends from the body to the tube with the air vent.

4. The nasal irrigator of claim 1, wherein the connection mechanism is connected to the vessel by screw-fastening.

5. The nasal irrigator of claim 1, wherein the connection mechanism is a bung.

6. The nasal irrigator of claim 1, wherein the rinsing liquid is mineral water.
